(19) United States
(12) Patent Application Publication
(10) Pub. No.: US 2018/0229006 A1
(43) Pub. Date: Aug. 16, 2018

(54) A MEDICAL TUBE STORAGE SYSTEM

(71) Applicant: Tookie Limited, Merseyside (GB)

(72) Inventor: Stephen Tooke, Merseyside (GB)

(21) Appl. No.: 15/751,892

(22) PCT Filed: Aug. 2, 2016
(86) PCT No.: PCT/GB2016/052375

§ 371 Date: Feb. 12, 2018

(30) Foreign Application Priority Data
Aug. 28, 2015 (GB) 1515389.3

(51) Int. Cl. A61M 25/02

(52) U.S. Cl. CPC A61M 25/02; A41D 13/1245; A61M 2205/0205; A61M 2025/0206

(57) ABSTRACT

A medical tube holder (10) comprising a harness (11) and a pouch (26). The harness comprises a front panel (12) and a back panel (14) with two shoulder straps (16a, 16b) connecting an upper part of the front panel to an upper part of the back panel, with a head aperture (18) between the shoulder straps. The lower part of back panel comprises a first part of a connection mechanism (22) and the lower part of the front panel comprises two laterally extending straps (20a, 20b). The two laterally extending straps form a second part of the connection mechanism and the pouch is connected to one of the laterally extending straps.

### FIELD OF THE INVENTION

The invention relates to a medical tube holder, particularly for use in patients with a long-term tube implanted.

### BACKGROUND TO THE INVENTION

It is well-known to implant medical tubes into patients and to have one end of the tube protruding from the patient. For example, central venous access catheters (“central lines”), and in particular so-called “Hickman lines”, are often required to be fitted in patients for extended periods. These lines are frequently used to administer medication or fluids and to obtain blood from a patient for testing. The central lines may be kept in place for several months at a time.

There are various complications with such central lines, including bloodstream infection due to bacteria ingress through the central line, which can be particularly damaging in cancer patients that may have become immunocompromised as a result of chemotherapy.

The risk of bacterial infection is particularly prevalent in infants and children that are less inclined to take care to keep the central line clean. Additionally, younger patients may be more prone to mechanical damage to the central line by, for example, catching it on objects or pulling at it.

The same applies to animals that may have medical tubes implanted long-term that protrude from the patient.

Whilst previously it has been known to affix the end of the tube to the patient's skin, for example with medical tape, the end of the tube is still exposed to bacteria and the tape can become irritating, leading the patient to scratch at it, thereby increasing the risk of mechanical damage to the tube and the patient.

### SUMMARY OF THE INVENTION

Accordingly, the present invention is directed to a medical tube holder comprising a harness and a pouch, the harness comprising a front panel and a back panel with two shoulder straps connecting an upper part of the front panel to an upper part of the back panel and a head aperture between the shoulder straps, wherein the lower part of back panel comprises a first part of a connection mechanism and the lower part of the front panel comprises two laterally extending straps, the two laterally extending straps forming a second part of the connection mechanism and wherein the pouch is connected to one of the laterally extending straps.

By providing a harness with a pouch to a patient, the patient is provided with a convenient storage location for the end of a medical tube, for example a central line, which can be worn. Such a harness thus provides a safe and secure manner in which to store the end of a protruding medical tube that moves with the patient.

The use of laterally extending straps keeps the device held on the patient and reduces the likelihood of it moving relative to the patient's torso. This is particularly useful when the patient is sleeping. The laterally extending straps in combination with the shoulder straps also reduce longitudinal movement of the tube relative to the patient. Additionally, the laterally extending straps provide a convenient location to which items may be attached.

An advantage of the harness being back-fastening, is that young children that might otherwise be inclined to remove the harness, find it more difficult to release connection mechanism and remove it. Therefore, the end of a medical tube can be stored in the pouch with a reduced risk that the child will remove the device without first removing the end of the medical tube, thereby reducing the risk of mechanical damage.

Preferably, the pouch comprises a liner. The use of a liner, and in particularly a removable liner that is anti-bacterial and anti-microbial, allows the end of the tube to be wrapped in the liner to reduce the risk of it being exposed to potentially harmful bacteria. Additionally, where the liner is removable from the pouch, the end of the line can be swiftly located by removing the liner from the pocket, which may be particularly useful in emergency situations. Additionally, the liner may be disposable and so can be readily replaced after each use, thereby further reducing the risk of infection.

Advantageously, the pouch is releasably connected to one of the laterally extending straps. The pouch being positioned on one of the laterally extending straps provides a convenient location for storing the end of the tube. The pouch may comprise a loop through which the strap can pass or it may comprise one of a number of connection means, for example, a hook-and-eye fastener, a snap fastener or a zipper mechanism. By allowing the pouch to be removable, it can be replaced and/or washed, when required. Additionally, it can be put on either side of the patient, thereby enabling it to be moved if it causes discomfort, for example during sleep.

In one arrangement the first part of the connection mechanism comprises one or more apertures or substantially vertical slits through which the laterally extending straps can pass. The use of slits in the lower part of the back panel provides a simple mechanism for securing the laterally extending straps to the back panel. Whilst it is envisages that one could apply loops to the back panel through which the laterally extending straps may pass, the use of the back panel itself, and apertures therein, reduces the number of parts and allows one to use the same material for the back panel and connection mechanism, thereby reducing the need to attach further parts. This increased the comfort of the harness, particularly when the user is sleeping because fewer seams and no hard plastics parts are required.

It may be advantageous that the lower part of the back panel comprises a flap and wherein that flap can be folded over the first part of the connection mechanism, thereby sandwiching the laterally extending straps when then engage the first part of the connection mechanism.

It may be further advantageous that the laterally extending straps and/or the flap comprise a releasable retaining connection and, preferably, the releasable retaining connection comprises one or more selected from group comprising: hook-and-eye fastener; a popper; and a button. The use of a flap to cover over where the laterally extending strap pass through the back panel reduces the risk of the mechanism releasing. This is particularly true where the flap is also provided with one part of hook-and-eye connection system and the back panel and/or the reverse of the laterally extending straps are also provided with the other part of such a connection system.

It is preferred that the holder comprises at least one anti-bacterial and/or anti-microbial material selected from a group comprising: polyester warp spacer fabric; paper; and bamboo yarn. The device may be manufactured from materials that are naturally anti-bacterial and/or antimicrobial, or they materials may be treated so that they are.

The invention extends to a method of using the device herein described.

### BRIEF DESCRIPTION OF THE DRAWINGS

An embodiment of the invention will now be described, by way of example only, and with reference to the accompanying drawings, in which:

FIG. 1 is a drawing showing an unassembled device in accordance with the present invention;

FIG. 2 is a drawing of a perspective view of the device of FIG. 1 in an assembled state;

FIG. 3 is a drawing of a rear view of the device of FIG. 2;

FIG. 4 is a drawing of a side view of the device of FIG. 2;

FIG. 5 is drawing of a second perspective view of the device of FIG. 2.

FIG. 6 is a front view of the device of FIG. 2.

### DETAILED DESCRIPTION OF EXEMPLARY EMBODIMENTS

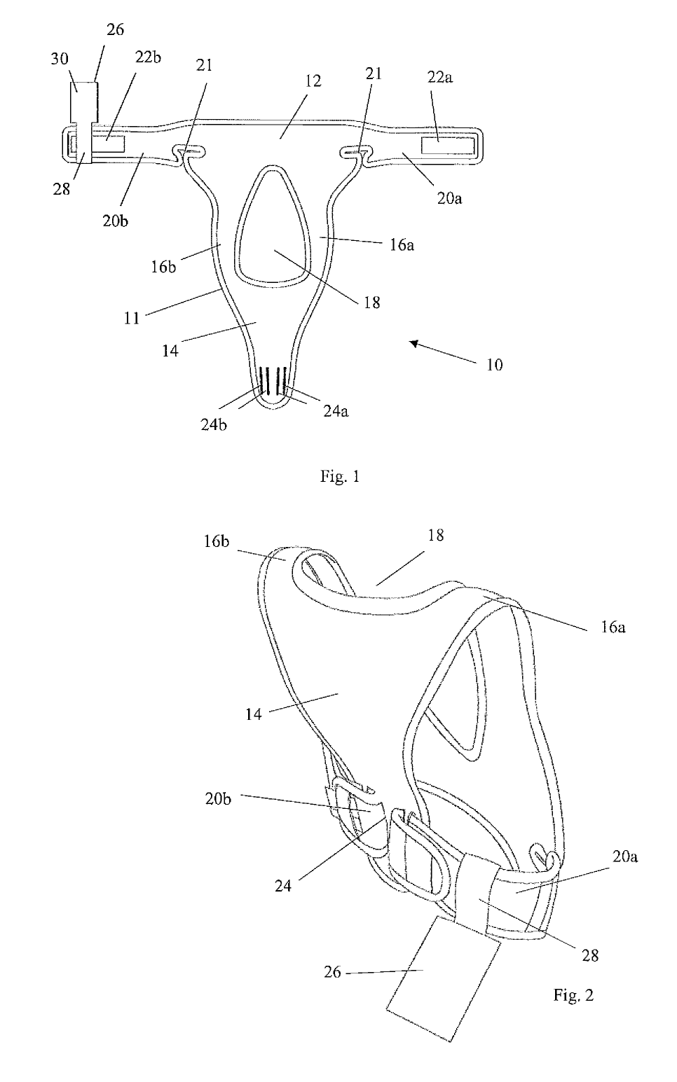

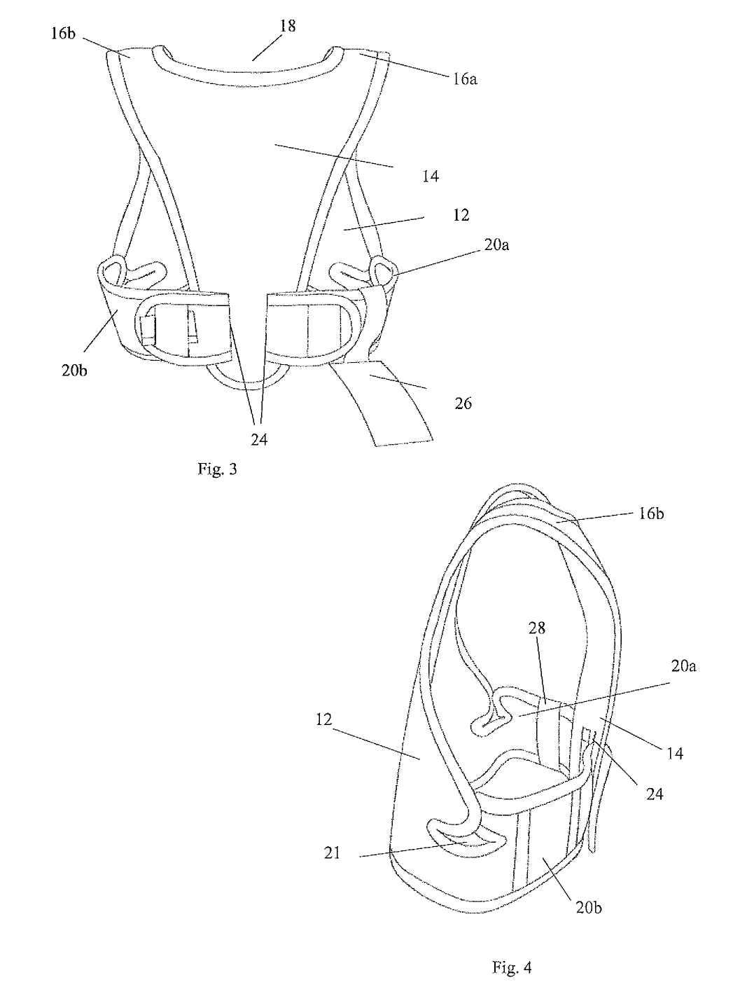

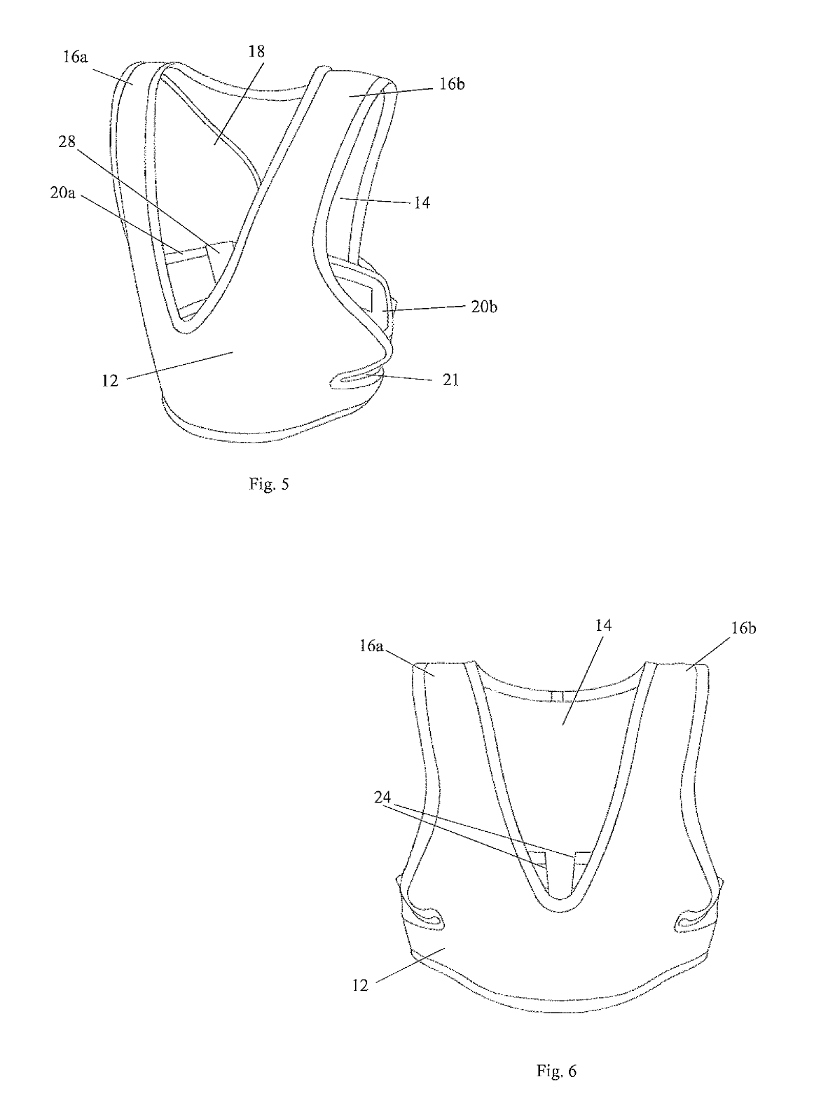

FIGS. 1 to 6 show a medical tube holder 10 in the form of a harness 11. The harness 11 comprises a front panel 12 and a back panel 14 with shoulder straps 16a and 16b connecting the tops of the respective panels 12 and 14. A head aperture 18 is formed between the two straps 16 and the front panel 12 and the back panel 14.

Connected to the lower part of the front panel 12, are two laterally extending straps 20a and 20b that extend substantially perpendicularly to the front panel 12, although the angle may vary, and that, in use, extend laterally about the torso of a patient. The ends of the two laterally extending straps 20a and 20b are provided with a connection material 22 comprising a portion of hook material at one end and a portion of eye material at the other, thereby constituting the two parts of a hook-and-eye connection. A recessed area 21 is arranged where the strap 20 connects with the front panel 12

The lower part of the back panel 14 is provided with four slits 24, positioned in two pairs either side of the centre of the back panel 14.

A pouch 26 is provided comprises a looped portion 28 and a receiving pocket 30, is threaded onto one of the straps 20b, with the strap 20b passing through the looped portion 28 of the pouch 26. The pocket 30 is provided with a removable and disposable liner (not shown). The ends of the liner may extend from the pocket 30 to allow quick access to the end of the tube by pulling the liner from the pocket 30 and thus exposing the end of the tube.

In use, the head aperture 18 is passed over a user's head such that the shoulder straps 16 rest upon the user's shoulders. The laterally extending straps 20 are then passes about the user's sides. The ends of each strap 20 pass through a respective and they pass through a first of the slits 24, the first slits being the ones closest to the middle of the back panel sagittally (when worn). The ends of the straps 20 then pass through the respective adjacent second slits 24 and overlay themselves. The hook portion of the connection material 22 is pressed onto the eye material of the connection material 22 and the straps 20 are held in place. Where a flap (not shown) is employed, the flap folds over the top of the slits 24 to provide further security to the straps 20 and to reduce the risk of them disengaging the back panel 14. When the device 10 is worn, the connection system (straps 20 and slits 24) are positioned at the rear of the user.

The end of the tube extending from the user can then be stowed in the pouch 26. Additionally, the tube can pass through the recessed area 21 to assist with supporting it in place and reducing the risk of longitudinal movement.

A second pouch may be connected to the device for holding pouch liners and/or antibacterial wipes. Other pouches and/or pockets may be provided.

The harness may comprise a paper material and it may be disposable. Alternatively, the device may be manufactures from textiles and it may be washable for long-term use.

1. A medical tube holder comprising a harness and a pouch, the harness comprising a front panel and a back panel with two shoulder straps connecting an upper part of the front panel to an upper part of the back panel and a head aperture between the shoulder straps, wherein the lower part of back panel comprises a first part of a connection mechanism and the lower part of the front panel comprises two laterally extending straps, the two laterally extending straps forming a second part of the connection mechanism and wherein the pouch is connected to one of the laterally extending straps.

2. A holder according to claim 1, wherein the pouch comprises a liner.

3. A holder according to claim 1, wherein the pouch is releasably connected to one of the laterally extending straps.

4. A holder according to claim 1, wherein the first part of the connection mechanism comprises one or more substantially vertical slits through which the laterally extending straps can pass.

5. A holder according to claim 1, wherein the lower part of the back panel comprises a flap and wherein that flap can be folded over the first part of the connection mechanism, thereby sandwiching the laterally extending straps when then engage the first part of the connection mechanism.

6. A holder according to claim 5, wherein the straps and/or the flap comprise a releasable retaining connection.

7. A holder according to claim 6, wherein the releasable retaining connection comprises one or more selected from group comprising: hook-and-eye fastener; a popper; and a button.

8. A holder according to claim 1, wherein the holder comprises at least one anti-bacterial and/or anti-microbial material selected from a group comprising: polyester warp spacer fabric; paper; and bamboo yarn.
